(19) United States
(12) Patent Application Publication
(10) Pub. No.: US 2006/0025377 A1
(43) Pub. Date: Feb. 2, 2006

(54) Inhibitor of angiogenesis and kit for treating cancer comprising the inhibitor

(76) Inventors: Chang Seo Park, Gwacheon-city (KR); Jin Wook Kim, Yong-in city (KR); Ki-Beom Suhr, Daejeon-city (KR); Chang-Hee Lee, Daejeon-city (KR); Jeong-Hoon Lee, Daejeon-city (KR); Jang-Kyu Park, Daejeon-city (KR)

Correspondence Address: DARBY & DARBY P.C., P. O. BOX 5257, NEW YORK, NY, 10150-5257, US

(21) Appl. No.: 10/537,319

(22) PCT Filed: Dec. 1, 2003
(86) PCT No.: PCT/KR03/02622

§ 371 Date: Jun. 1, 2005

(30) Foreign Application Priority Data
Dec. 2, 2002 (KR) 10-2002-0076022

(51) Int. Cl. A61K 31/685; A61K 31/7008

(52) U.S. Cl. 514/54; 514/78

(57) ABSTRACT

The inhibitor of angiogenesis is characterized by containing tetraacetylphytosphingosine and the kit for inhibiting angiogenesis is characterized by the inhibitor. The inhibitor and kit are effective in treating and preventing disorders in connection with extremely increased angiogenesis such as angioma, tumor and psoriasis by effectively inhibiting angiogenesis. Furthermore, they are effective in inhibiting proliferation of cancer cells and metastasis of cancer without any adverse effect.

### TECHNICAL FIELD

The present invention relates to the inhibitor of angiogenesis comprising tetraacethylphytosphingosine derivatives and the kit for treating cancer comprising the inhibitor.

### BACKGROUND ART

A process called angiogenesis which was started to be researched with observation of new formation of blood vessels in placenta in 1935 was found in various fields such as nidation, developing child in a mothers womb, wound healing, menstruation for women, arthritis, diabetic retinopathy or the like. As accumulating evidences have been found that there are many blood vessels and frequent bleeding around cancer cells and angiogenesis plays key roles in development of cancer and the growth and metastasis of cancer cells, various researches on the substances which inhibit angiogenesis are under progression. As a results of full-scale researches on angiogenesis which was carried out from the 1960s, the fact that the cause of the rapid proliferation of cancer cells is angiogenesis was found, and angiogenic factors started to be found in 1980s. A variety of angiogenesis inhibition factors and angiogenesis inhibitors were found in 1990s, and their potentiality as an inhibitor of cancer cell proliferation was expanded drastically. Nowadays, many anti-angiogenic strategies are being evaluated in clinical trials. These approaches offer new hope for the successful treatment of cancer.

For develpomant and proliferation of cancer cells, first growth phase related to tumor growth factors is observed. In this phase, various tumor growth factors and blood vessels formation factors are expressed significantly, and cancer cells and new blood vessels are formed. Following the growth phase, a phase that infiltration of cancer cells occurs starts. An disequilibrium between proteinases which digest extracellular matrix and basement membrane, and inhibitory substances of proteinases occurs in this phase, wherein proteinases such as MMP-2 (matrixmetalloproteinase-2), MMP-9 (matrixmetalloproteinase-9), uPA (urokinase type plasminogen activator) or the like increas, whereas PAI-1 (plasminogen activator inhibitor-1), TIMP (tissue inhibitor of metalloproteinase) or the like involved in inhibition of proteinases decrease. Finally, in a phase that metastasis of cancer cells occurs, activity of cell adhesion molecule increases, thereby increasing adhesiveness of cells and metastasis of cancer cells occurs completely. Since such changed biological activities occurred in development and metastasis of cancer can be regulated by each of specific inhibitors, various studies on biological treatments based on above concepts are set about actively, and improvements of treatment effect through selective treatment are expected.

First, infiltration of cancer cells and angiogenesis require proteinases. Cancer cells, fibroblasts and endothelial cells produce proteinases and decompose extracellular matrix and basement membrane to give rise to infiltration of cancer cells and angiogenesis. Proteinases involved in such process include serine protease and MMPs (metrixmetalloproteinase). In process that such proteases decompose extracellular matrix, uPA (urokinase type plasminogen activator) converts plasminogen into plasmin to destroy fibrin, fibronectin, proteoglycan, laminin around cancer cells, and activates collagenase to decompose collagen. However, since uPA is inhibited by PAI-1 (plasminogen activator inhibitor-1), PAI-1 is expected to control abilities of angiogenesis and metastasis of cancer cells.

In cancer cells, MMP-2 and MMP-9 are primarily activated. MMP-2 is activated by MMP presented in cell membrane of cancer cells, whereas MMP-9 is inhibited by TIMP. Therefore, recently it is expected that controlling the disequilibrium of MMP and TIMP is used effectively to inhibit formation of blood vessels, metastasis and infiltration, and thus it is suggested as a novel treatment concept.

When cancer cells are not supplied with nutrients through the formation of blood vessels, their growth is limited. Also, newly formed blood vessels are major network for metastasis in addition to channel for suppliment of nutrients. The relationship between the degree of tumor angiogenesis and metastasis was well known, and it is established in various cancer cells that capillaries density of in situ tumor plays a key role in expectation of prognosis and metastasis of cancer. Recently, since monoclonal antibodies directed to angiogenic factors were developed, the degree of angiogenesis could be determined by measuring directly of angiogenic factors such as bFGF, VEGF, TGF-b or the like.

The concepts of cancer treatment through inhibition of angiogenesis are that connection of biological changes which are disturbed due to cancer as compared cancer cell with normal cell is to prevent differentiation, proliferation and metastasis of cancer cell, and as a result to block growth of cancer. Based on such concepts, MMP inhibitors have been synthesized and used for treating cancer since 1980s. However, according to the results of clinical tests, the effect of MMP inhibitors is far below what are expected This is because the clinical tests are carried out on the subjects of which cancer has been already metastasized.

Angiogenesis is essential for proliferation and metastasis of tumor cells. Also, newly formed blood vessels are major network of metastasis of cancer cells. A proliferation and a migration of endothelial cells are events that occurred frequently in only cancer cells, except for wound healing for adults and menstruation for women. Consequently, angiogenesis that occurs in cancer tissues rather than normal tissues is a very selective target of cancer treatment, and in theory such treatment is considered to have little adverse effect and can be used together with other cancer treatments, thereby increasing effect of the treatment. As a result, substances to inhibit effectively angiogenesis can be used effectively to treat disorders in connection with extremely increased angiogenesis such as angioma, tumor and psoriasis.

### DISCLOSURE OF THE INVENTION

An object of the present invention is treating and preventing disorders in connection with extremely increased angiogenesis such as angioma, tumor and psoriasis by effectively inhibiting angiogenesis. Furthermore, other object of the present invention is providing a pharmaceutical composition to inhibit proliferation of and metastasis of cancer cells effectively without any adverse effect.

To achieve above objects, the inhibitor of angiogenesis of the present invention is characterized by containing tetraacetylphytosphingosine.

Also, the kit for treating cancer of the present invention is characterized by comprising the inhibitor of angiogenesis containing tetraacetylphytosphingosinethe.

The kit for treating cancer is characterized by further comprising anti-cancer drug and irradiator.

For the kit for treating cancer, the anti-cancer drug is characterized by spingolipid derivatives.

For the kit for treating cancer, the spingolipid derivatives are one or more spingolipid derivatives selected from the group consisting of phytosphingosine, enacethylphytosphingosine, C6 phytocerarnide, C8 phytosphingosine, dimethylsphingosine, dimethylphytosphingosine and sphingosine.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a graph representing the number of blood vessels determined on 4 days after treatment of 0.1 μM, 1 μM, 2 μM and 5 μM of a solution containing tetraacetylphytosphingosine according to the present invention respectively in comparison with negative control.

FIG. 2 is a graph representing the area of granulation tissue determined on 4 days after treatment of 0.1 μM, 1 μM, 2 μM and 5 μM of a solution containing tetraacetylphytosphingosine according to the present invention respectively in comparison with negative control.

FIG. 3 is a graph representing a result of toxicity test of a solution containing tetraacetylphytosphingosine according to the present invention on HUVEC cell.

FIG. 4 is a graph representing a result of angiognesis test of a solution containing tetraacetylphytosphingosine according to the present invention.

FIG. 5 is photographes representing a result of angiognesis test of a solution containing tetraacetylphytosphingosine according to the present invention.

FIG. 6 is photographes representing that a solution containing tetraacetylphytosphingosine according to the present invention inhibited migration of endothelial cells.

FIG. 7 is a graph representing that a solution containing tetraacetylphytosphingosine according to the present invention inhibited migration of endothelial cells.

### BEST MODE FOR CARRYING OUT THE INVENTION

The present invention will now be described in more detail.
the inhibitor of angiogenesis and the kit for treating cancer comprising the same according to the present invention are described below in detail.

A composition according to the present invention comprises sphingolipid to inhibit angiogenesis very effectively. In particular, We found that acethylated derivatives of phytosphingosine, tetraacetylphytosphingosine inhibits angiogenesis and migration of HUVEC(Human Umbilical Vein Endothelial Cell) cell line strongly and it is effective in treating disorders in connection with extremely increased angiogenesis such as angioma, tumor and psoriasis by effectively inhibiting angiogenesis.

Angiogenesis in malignant tumor plays key roles that supply cancer cell with nutrients to allow cancer cell to grow rapidly, concurrently that function as migration network for metastasizing cancer cell to another tissues and organs. Meanwhile, A number of newly formed blood vessels are observed in hyperkeratosis region of skin diseases such as psoriasis. In this respect, it is believed that an inhibitor of angiogenesis inhibits the growth of cancer cells and prevents the metastasis of cancer cells as well as has little adverse effect. Therefore, because it is expected that a inhibitor of angiogenesis increases the effect of cancer treatment significantly, many studies have been focused on a inhibitor of angiogenesis. Also, it is expected that when a inhibitor of angiogenesis is used in conjunction with various anticancer treatments in patients who have obvious goal of a treatment and is expected to take the effect of the treatment, the effect of treatment will be excellent.

Sphingolipid is well known as sunstances involved in signal transduction in cell and play an important role in proliferation, differentiation and apoptosis or programmed cell death of cell. Ceramide is a kind of sphingolipid, and is sphingosine-based signaling molecules that fatty acid is linked to sphingosine backbone. ceramide is known as 2nd messenger involved in stress signaling, senescence, cell death, etc. Ceramide is generated from hydrolysis of membrane sphingomyclin that enriched in the brain on receiving the signal from TNF-alpha, Fas, etc. and determine the destination of cell. Sphingomyelin is converted into ceramide by sphingomyelinase, and subsequently it is converted into sphingosine by removing the fatty acid from ceramide by ceramidase. Sphingosine is converted into sphingosine-1-phosphate by sphingosine kinase, and subsequently it is decomposed by lyase. It is known that ceramide and long chain base of sphingosine give rise to apoptosis, and sphingosine-1-phosphate that phosphate group is linked to sphingosine make a function to stimulate cell growth and cell proliferation. Since the balance of sphingosine and phosphated sphingosine within cell is involved in cell growth, cell proliferation and cell death, the change of sphingolipid concentration affects cell lethally. It is well known that most anticancer drugs have a mechanism that causes to apoptosis by increasing the amount of ceramide within cell. Also, It is known that since cancer cells have a different metabolic pathway of ceramide as compared with normal cells, and thus the concentration of ceramide of normal cells is lower than that of cancer cell apoptosis is not occur in cancer cells, and cancer cells can grow and proliferate rapidly. it has been found in various studied that ceramide and long chain bases of shhingosine cause cancer cells to apoptosis. Recently, It was found that various derivatives of phytosphingosine that is sphingolipid and is produced in yeast causes cancer cells to apoptosis.

Many studies have been performing which attempt to give rise to apoptosis of cancer cell by regulating the metabolism of cermide. First, various chemotherapies and radition therapies which are carried out on cancer cells cause to formation of ceramide within cancer cells and as a result give rise to apoptosis of cancer cells. Also, other approach which gives rise to apoptosis of cancer cell is direct treatment of ceramide and long chain bases of sphingolipid to cancer cells. Further, still other approach which can be used effectively is to block the decomposition of ceramide by administering ceramidase inhibitor for preventing the decrease of ceramide contents, or to block conversion of sphingosine into sphingosine-1-phosphate by administering sphingosinekinase inhibitor such as DMS (dimethylsphingosine) for inhibiting the growth of cancer cells.

Sphingosine-1-phosphate is in large amount in blood and plays a role that transduces signals of various foreign responses. It is known that when blood vessels are injured due to wound, sphingosine-1-phosphate in blood that stimulates formation and migration of endothelial cells is secreted in large amount to heal wound rapidly. Also, it is a well known fact that SPC (sphingosinephosphorylcholine) which is shpingosine derivatives has a excellent effect on wound healing.

The effect of long chain bases of sphingosine to kill cancer cell, which were found in precedent various studies represents that sphingosine can be used effectively for treating cancer. However, substances that inhibit migration of endothelial cells and angiogenesis, and concurrently give rise to apoptosis are expected to have better effect to inhibit growth of cencer cells and give rise to apoptosis.

Recently, it has been reported frequently that phytosphingosine produced in yeast like sphingosine give rise to apoptosis of cancer cell. It was found that derivatives of phytosphingosine(phytosphingosine, N-acethylsphingosine, tetraacethylsphingosine, C6 phytoceramide, C8 phytoceramide or the like) give rise to apoptosis in various cell lines including HaCat (keratinocyte), fibroblast, CHO(Chinese hamster ovarian cell), HL-60 (Human leukemia), B16F10 (Melanocyte cell line), U937 (Monocyte) or the like and cancer cell lines (H460, A539: lung cancer). Further, it is believed that phytosphingosine derivatives are involved in various inflammatory responses because of their inhibitory effect of protein kinase C and phospholipase D.

A compound of the present invention can be administered by any means that achieve its intended purpose. For example, administration can be by oral, parenteral, rectal, vaginal, topical, intravenous, intramuscular, intraperitoneal, subcutaneous or the like. The dosage administered of the active compound will be dependent upon the recipient, particular diseases or pathological states to be treated, the severity of diseases or pathological states, mode of administration and judgement of prescriber. A determination of the dosage based on the above factors is well known to those skilled in the art. Generally, the dosage is within range of about 0.01 mg/kg/day to about 2000 mg/kg/day, preferably 0.5 mg/kg/day to 2.5 mg/kg/day.

The compound of the present invention can be formulated into pharmaceutical compositions with suitable pharmaceutically acceptable carriers. The pharmaceutical compositions are manufactured in a manner that is, itself, kiown by using typical carriers (see, for example, E. W. Martin, Remington's Pharmaceutical Sciences, latest edition, Merck Publ. Co., Easton, Pa.). The compound of the present invention can be administered together with other anticancer drugs. Also, the pharmaceutical compositions can be administered in conjunction with other agents and treatments for treating diseases. For example, when the pharmaceutical compositions are administered, surgery, radiation therapy or chemotherapy can be also carried out concurrently. According to the intended mode of administration, the pharmaceutical compositions can be in the forms of solid, semi-solid or liquid. The dosage forms include, but not limited to tablets, pills, capsules, suppositories, granules, small saccus, powders, creams, lotions, ointments, patches, liquid solutions, suspensions, dispersions, emulsions, syrups or the like. Also, the active ingredients can be encapsulated in liposome, microparticle or microcapsule.

General nontoxic carriers include, but are not limited to mannitol, lactose, starch, magnesium stearate, sodium saccharine, talc, cellulose, glucose, sucrose, dextrose, glycerol, magnesium carbonate, triglyceride, oil, solvent, sterile water, isotonic saline (pharmaceutical grade) and the like. Solid composition such as tablets, pills, granules or the like can be coated conveniently.

Typically, composition for administrating intraveneously is a solution in sterile isotonic buffer and contains topical anesthetic for alleviating pain at injection site. If desired, drug can contain small amount of nontoxic auxiliaries such as wetting agent, emulsifier, pH buffer and the like. Examples of such auxiliaries include, but are not limited to sodium acetate, sorbitan monolaurate, triethanolamine and triethanolamine oleate. Also, the composition according to the present invention comprises excipients such as stabilizer, antioxidant, binder, colorant, flavoring agent and thickening agent.

The inhibitor of angiogenesis according to the present invention comprises tetraacetylphytosphingosine, preferably 0.001 percent by weight to 99 percent by weight of tetraacetylphytosphingosine based on total composition. Less than 0.001 percent by weight of tetraacetylphytosphingosine has little effect, and 99 percent by weight or less represents that there is other additives or impurities in the composition.

The present invention will now be illustrated by the following preferred embodiments, but not limiting the scope of the invention.

### EXAMPLES

We established that tetraacetylphytosphingosine caused cancer cell to apoptosis, and concurrently inhibited angiogenesis by determining apoptosis and inhibitory effect of angiogenesis of tetraacetylphytosphingosine as described below, and thus it can be used effectively for treating cancer.

### Example 1

### In Vivo Wound Healing Assay

Female New Zealand White rabbits (body weight 2.0 kg) were used as experimental animals in this assay. PBS solution containing 0.1% BSA (BSA-PBS solution) was prepared as control solution for negative control group (comparative example 1). sphingosylphosphorylcholine (comparative example 2), phytosphingosine (comparative example 3), N-acethylphytosphingosine (comparative example 4) as positive control group, and tetraacetyl phytosphingosine (example 1) were dissolved in ethanol or methanol respectively. Each portion of the solutions was added to silicone glass tube. Then, it was charged with N₂ gas, and added 0.1% BSA-PBS solution. After coupling them with water sonicator and vortex, these solutions were spotted on and injected intradernally to wound region of experimental animals. At the same time, to study an effect of the angiogenesis as concentrations of tetraacethylsphingosine, the animal was treated with 0.1 μM, 1 μM, 2 μM and 5 μM of tetraacethylsphingosine, and campared with the results. Experimental animals were put into special stainless cages which was designed suitably to assay experimental animals, anesthetized by injecting ketanine (3-4 mg/kg) intramuscularly. The hair and corneous tissue of innerside of both ears were removed with shaving and washing, and then were disinfected with 70% ethanol. Four wound regions per ear were formed by using 6 mm punch for sldn histological examination (Stiefel, Germany) under sterile condition if possible, and each wound region was spotted or injected intradennally 30-50 μl of the control solution or each treatment material. Wound regions were sealed with cathereep (Nichiban Co., Tokyo Japan) which was cut in size which are greater than that of wound region to prevent contamination of the wound regions and formation of crust. Then, the wound regions were protected with 2×2 gauze, and ears of rabbit were bandaged with elastopore (Nichiban Co., Tokyo Japan). Subsequently, the rabbits were bred in a cage per a rabbit. After 48 hrs, same procedure was repeated. On 4 days and 8 days after forming wound regions, sacrificing the rabbits and treating tissue for histological study. For histological study, wound tissues were fixed with 10% formalin, cut it in half longitudinally and made paraffin block. Then, approximately 5 μm of segment was made, attached it to slide and stained with hematoxylin and eosin to observe a change of epidermis and dermis, and stained with Massons Trichrome to observe a degree of collagen formation of granulation tissue.

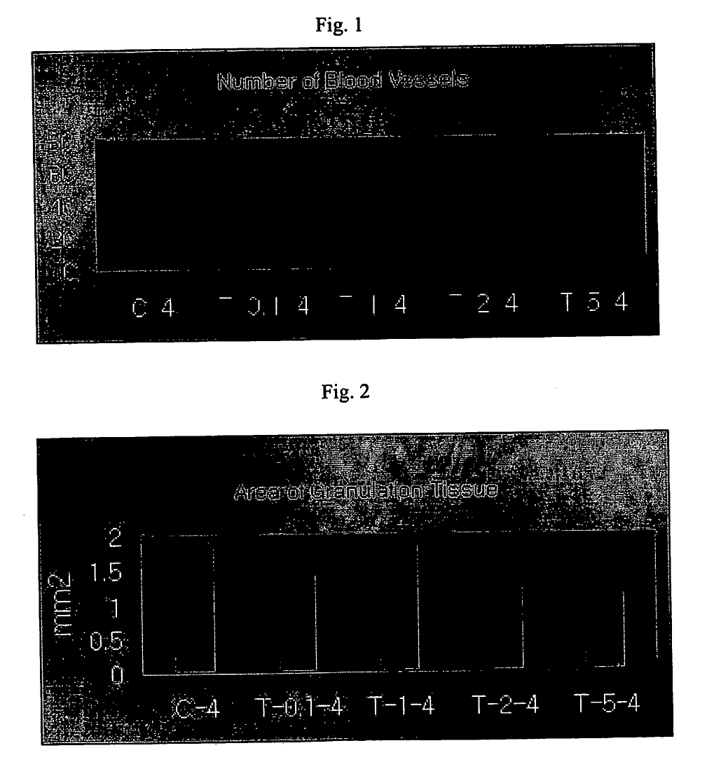

First, stained tissue specimen was calibrated with ocular micrometer of optical microscope for image analysis. Then, After photographing each histological change aspects with digital camera under 40× and 100× objective lens and saving them on computer (Pentium III), image analysis was carried out with Scion Image for Windows software which was provided by (C) 2000 Scion corporation as follows: that is, degree of epidermis migration could be determined by measuring the distance from left to right boundary of wound, and thickness of newly formed epidermis could be determined by measuring the thickness of three spots in 1 mm of pitch and averaging measured values. To compare with degree of granulation tissue formation of dermis, three methods were used: 1) A method of measuring total area of newly formed granulation tissue and comparing with it; 2) A method of counting the number of cells such as fibroblast presented in six spots of center region of wound under high magnification (100×) and comparing with it; and 3) A method of determining degree of angiogenesis by counting the number of capillaries in granulation tissue in a same way as described above. If there was eschar, it wasn't determined. The data obtained from the negative control group, the positive control group and the experinental group were analyzed statistically by carrying out paired student's t test. The results were shown in FIG. 1, FIG. 2 and Table 1.

FIG. 1 is a graph representing the number of blood vessels determined on 4 days after treatment of 0.1 μM, 1 μM, 2 μM and 5 μM of a solution containing tetraacetylphytosphingosine according to the present invention respectively in comparison with negative control. In FIG. 1, C-4 represents a result of a negative control group which elapsed 4 days after treatment. T-0.1-4 represents a result of a experimental group which elapsed 4 days after treatment of 0.1 μM of tetraacethylphytosphingosine (TAPS). T-1-4, T-24 and T-54 represent results of experimental groups which elapsed 4 days after treatment of 1 μM, 2 μM and 5 μM of TAPS respectively. As shown in FIG. 1, tetraacethylphytosphingosine inhibited angiogenesis greatly.

FIG. 2 is a graph representing the area of granulation tissue determined on 4 days after treatment of 0.1 μM, 1 μM, 2 μM and 5 μM of a solution containing tetraacetylphytosphingosine according to the present invention respectively in comparison with negative control. In FIG. 2, C-4 represents a result of a negative control group which elapsed 4 days after treatment, and T-0.14, T-1-4, T-2-4 and T-5-4 represent results of experimental groups which elapsed 4 days after treatment of 0.1 μM, 1 μM, 2 μM and 5 μM of TAPS respectively. As shown in FIG. 2, a solution containing tetraacetylphytosphingosine according to the present invention decreased the area of granulation tissue.

Meanwhile, same experiments were carried out with 5 μM of the negative control group (comparative example 1), sphingosylphosphorylcholine (SPC)(comparative example 2), phytosphingosine (PS)(comparative example 3), N-acethylphytosphingosine (NAPS)(comparative example 4) and tetraacetylphytosphingosine (TAPS) as example (example 1) respectively, then the number of blood vessels and the area of granulation tissue were determined. The results were as follows:

Table 1

| | Comparative example 1 | Comparative example | Comparative example | Comparative example | Example 1: |
|---|---|---|---|---|---|
| | (negative control) | 2: SPC (5 μM) | 3: PS (5 μM) | 4: NAPS (5 μM) | TAPS (5 μM) |
| Area of granulation tissue | 100% | 151% | 112% | 212% | 76% |
| Number of blood vessels | 100% | 148% | 106% | 96% | 31% |

As shown in the Table 1, sphingosinephosphorylcholine (SPC) prompted increment of the number of blood vessels and the area of granulation tissue significantly, and tetracetylphytosphingosine (SPC) inhibited increment of the number of blood vessels and the area of granulation tissue significantly.

### Exemple 2

### Toxicity Test and Angiogenesis Test on HUVEC

HUVEC (human umbilical vein endothelial cell) which was used in this study was cultured as follows: Umbilical cord soaked in cold PBS were cut in 15-20 cm, and washing it thoroughly. Then, cannular was inserted into veins of umbilical cord at its both ends, and sutured umbilical cord and cannular tightly. 2-way stopcock was connected to each cannular which was tightly inserted to unbilical cord, and then 0.45 μm milipore filter was connected to one stopcock. After washing veins with PBS, collagenase solution was added, and incubated for 6 mins at 37° C. After 6 mins, collagenase solution was removed and harvested from veins by introducing heparin solution (10 ml) into the veins, and harvested cells were centrifuged at 1500 rpm for 5 mins. Precipitated endothelial cells were suspended in 5 ml of M199 medium that does not contain FBS. Above suspension procedure was repeated twice. Then, endothelial cells obtained in this manner were suspended in 5 ml of a medium which is suitable for growing endothelial cells, tmmsferred to T25 flask which coated with gelatin, and incubated in 5% CO₂ incubator at 37° C.

Meanwhile, human fibroblasts which were used in this study were incubated as follows: Skin tissues were obtained by circumcision sterilely, and washed three times with Hanks balanced solution to remove epidermis and subcutaneous fat. Dermis-tissues were cut in suitable size and placed them on the bottom of 35 mm culture dish. For depositing dermis tissues to the culture dish thoroughly, the culture dish was placed in incubator (5% CO₂, 37° C., Form a Scientific, Inc., Ohio, U.S.A.) for 5 mins, and then culture broth was added. After about 1-2 weeks, grown fibroblasts were treated with 0.25% trypsin solution and 0.02% EDTA solution for 3-5 mins, separated and passaged.

Toxicity of cell and growing capacity were determined as follows:

Various cell suspensions were stained with 0.5% Tryphan Blue to count cell numbers. Corresponding medium was loaded to column 1 of 96 multi well plate (blank). To all columns excluding column 1 was added dividedly 180 μl of cells conditioned in corresponding medium, and incubated in incubator at 37° C. for 12-24 hrs. 20 μl of a medicament that is 10% of culture broth was added to experimental group, and PBS in equal amount was added to control group. After further incubating for 2 days, 40 μl of MTT solution that the concentration had adjusted to 5 mg/ml with PBS (pH 7.4) was added, and further incubated in incubator at 37° C. for 4 hrs. And then the plate was centrifuged at 1500 rpm for 10 mins to discard the supernatant. 150 μl of 100% DMSO was added to the plate with multi-channel pipette, and the plate was shaked in plate shaker. Finally, the absorbance was determined with ELISA reader at 540 nm. Angiogenesis test of HUVEC was carried out as follows:

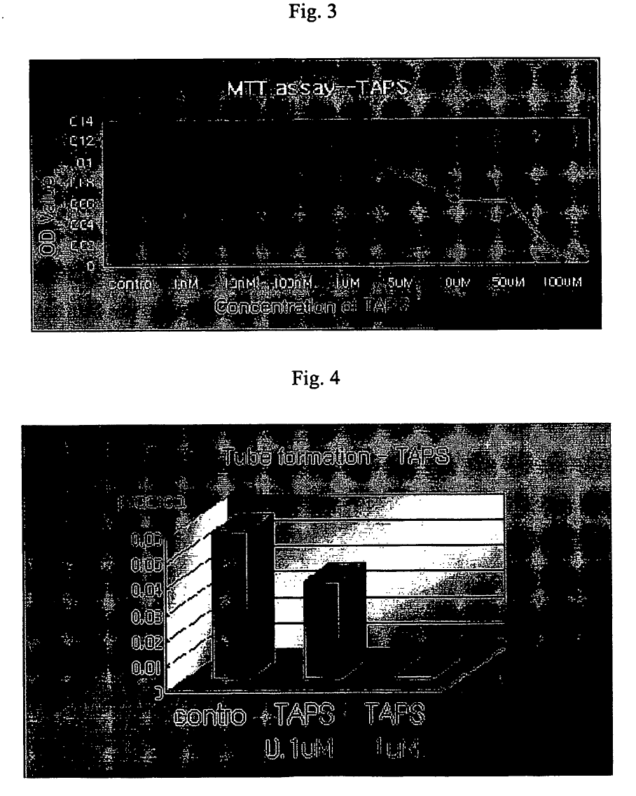

Matrigel was prepared as follows: First, collagen solution was prepared by mixing commercially available acid-soluble porcine type I collagen (3.0 mg/ml), 5× DMEM and buffer (0.05 N NaOH, 2.2% NaHCO₃, 200 mM HEPES; 7:2:1), loaded to 24 well or 96 well (100-300 μl/well) dividedly, and then incubated for approximately 10 mins to form a gel due to polymerization of collagen. Primary culture was carried to obtain HUVEC (those which were passaged five times), removed cells at 37° C., washed cells with PBS, detached cells with trypsin/EDTA, loaded cells to wells (4-6×10⁴ cells/well), and then fiuther incubated for 12-18 hrs. The results were shown in FIG. 3 and FIG. 4.

FIG. 3 is a graph representing a result of toxicity test of a solution containing tetracetylphytosphingosine according to the present invention on HUVEC cell. As shown in FIG. 3, apoptosis occurred at the concentration of 5 μM or more of TAPS dramatically.

FIG. 4 is a graph representing a result of angiognesis test of a solution containing tetraacetylphytosphingosine according to the present invention.

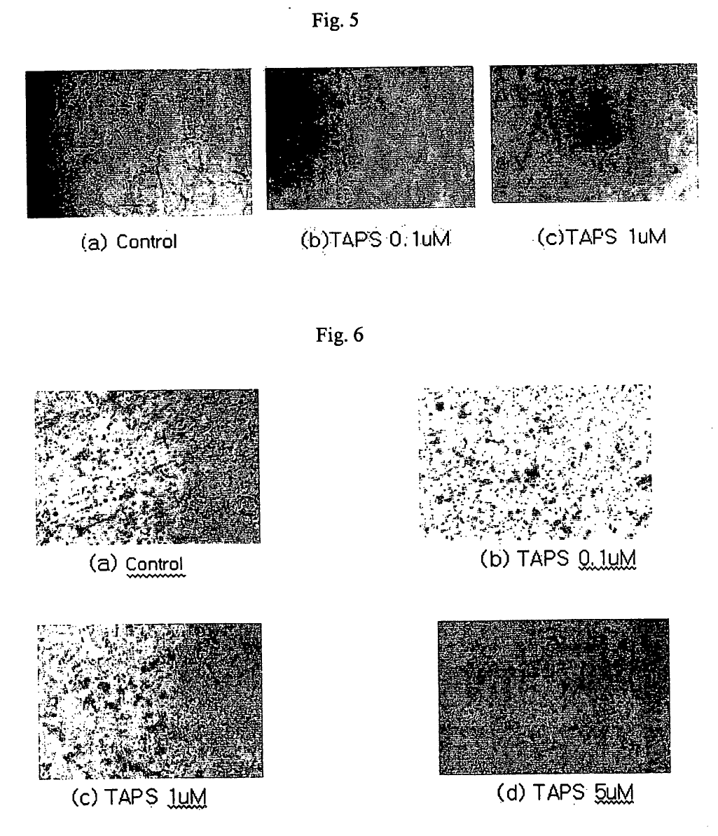

Also, FIGS. 5(a), 5(b) and 5(c) are photographs representing results of angiogenesis test of a solution containing tetraacetylphytosphingosine according to the present invention.

As shown in FIG. 4 and FIG. 5, for tube formation test, TAPS was inhibited effectively a formation of blood vessels at the concentration of 0.1 μM, and inhibited nearly completely a formation of blood vessels at the concentration of 1 μM.

### Example 3

### Migration Assay of Endothelial Cells

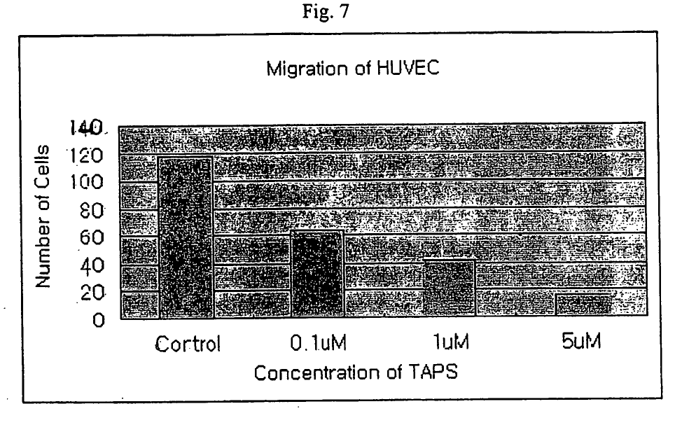

Transwell membranes were coated with 0.2% gelatin, and then were left for 12 hrs at 4° C. The membrane for control group was treated with BSA-PBS as described in the above examples, whereas the membranes for experimental group were treated with 0.1 μM, 1 μM and 5 μM of tetraacetylphytosphingosine solution according to the present invention respectively as described in the above examples. After incubating these membranes for 2 hrs, staining them with Diff Quick solution, and then observing them on slide glass. The results were shown in FIG. 6 and FIG. 7.

FIG. 6 is a graph representing that a solution containing tetraacetylphytosphingosine according to the present invention inhibited migration of endothelial cells.

FIG. 7 is photographs representing that a solution containing tetraacetylphytosphingosine according to the present invention inhibited migration of endothelial cells. As shown in FIG. 6 and FIG. 7, migration of endothelial cells were inhibited by TAPS, since the number of endothelial cells migrating to the opposite side of the membrane was reduced as increasing the concentration of TAPS.

The inhibitor of the present invention and the kit comprising it are effective in treating and preventing disorders in connection with extremely increased angiogenesis such as angioma, tumor and psoriasis by effectively inhibiting angiogenesis. Furthermore, they are effective in inhibiting proliferation of cancer cells and metastasis of cancer without any adverse effect.

Although preferred embodiments of the present invention have been described for illustrative purposes, those skilled in the art will appreciate that various modifications, additions and substitutions are possible, without departing from the scope and spirit of the invention as disclosed in the accompanying claims.

What is claimed is:

1. An inhibitor of angiogenesis containing tetraacetylphytosphingosine.

2. A kit for treating cancer, comprising an inhibitor of angiogenesis containing tetraacetylphytosphingosinethe.

3. The kit for treating cancer according to claim 2, wherein further comprising anti-cancer drug and irradiator.

4. The kit for treating cancer according to claim 3, wherein the anti-cancer drug is spingolipid derivatives.

5. The kit for treating cancer according to claim 4, wherein the spingolipid derivatives are one or more spingolipid derivatives selected from the group consisting of phytosphingosine, enacethylphytosphingosine, C6 phytoceramide, C8 phytosphingosine, dimethylsphingosine, dimethylphytosphingosine and sphingosine. A composition for inhibiting the activity of tyrosinase which comprises N-acetylphytosphingosine as an active ingredient.
